(19) United States
(12) Patent Application Publication
(10) Pub. No.: US 2013/0005740 A1
(43) Pub. Date: Jan. 3, 2013

(54) PROCESS FOR PREPARATION OF OVER-THE-COUNTER GELATIN OR PECTIN-BASED DRUG DELIVERY

(75) Inventors: Stuart A. Lowther, Burlington (CA); Real Plante, Upton (CA); Bradley H. Pilon, Dundas (CA); Mark A. Tarnopolsky, Hamilton (CA)

(73) Assignee: 7267207 CANADA LIMITED CORPORATION, Burlington, ON (CA)

(21) Appl. No.: 13/388,480

(22) PCT Filed: Aug. 5, 2010
(86) PCT No.: PCT/CA2010/001217

§ 371 Date: Sep. 19, 2012

Related U.S. Application Data
(60) Provisional application No. 61/231,627, filed on Aug. 5, 2009.

(51) Int. Cl. A61K 47/36; A61K 31/192; A61K 31/167; A61K 31/4545; A61K 31/495; A61K 31/137; A61K 31/138; A61P 25/04; A61P 29/00; A61P 37/08; A61P 11/14; A61P 11/02; A61P 31/00; A61P 11/10; A61K 47/42

(52) U.S. Cl. 514/255.04; 514/777; 514/774; 514/570; 514/630; 514/318; 514/653; 514/651

(57) ABSTRACT

Disclosed herein is a process producing a therapeutic comestible gummie product, the process comprises providing a sugar and/or sugar alcohol, water and heating to at least 80° C. to produce a first mixture. A gelling agent and water is also provided and heat to at least 80° C. to produce a second mixture. Combining the first and second mixtures to produce a slurry. The slurry is heated to at least 112° C. to produce a gummie base. The gummie base is cooled to an optimal temperature of below 90° C. for the addition of an active drug and the active drug is substantially uniformly mixed with the gummie base. In some embodiments the active is drug is mixed with an ingestible oil and added to the cooling slurry. The gummie base and active drug mixture is then poured into molding trays and allowed to cure. Also disclosed herein by way the process of the present disclosure is therapeutic comestible gummie product. Furthermore, the present disclosure comprises a therapeutic comestible gummie product comprising ibuprofen and/or other active drug(s).

### RELATED APPLICATION

The present application is related to and claims benefit of priority to U.S. Provisional Patent Application No. 61/231,627, filed Aug. 5, 2009, entitled “Process for Preparation of Over-the-Counter Gelatin or Pectin-Based Drug Delivery”, the entire subject matter of which is hereby fully incorporated herein by reference.

### FIELD OF THE DISCLOSURE

The present disclosure relates to a process for the manufacture of gelatin and pectin-based over-the-counter soft-chew or gummie-based medication. More specifically, the present disclosure relates to the product made by a process wherein over-the-counter medications are incorporated into a gelatin or pectin-based soft-chew or gummie-based candy for oral delivery.

### BACKGROUND OF THE DISCLOSURE

Children are noted for often getting sick. Their developing immune systems leave them susceptible to infections such as the common cold, the flu or suffering from allergies. And, as with adults, children suffer from headaches, fever, diarrhea, allergies, cough, pain and other assorted maladies for which over-the-counter medications may be useful. Although in the best interest of the child, parent and caregivers will attempt to give their children and charges over-the-counter medications in order to alleviate symptoms and make them feel better, however, children often and adamantly resist the administration of medications.

Moreover, young children often have trouble swallowing pills. As a result of this, many children's medications are produced in both solid, such a pill, or liquid versions. The liquid versions of children's over-the-counter medications seek to make it easier for a child to take the medications, however a major drawback with liquid medication is the taste. Many medications have very unpleasant tastes, often being bitter and as the liquid medication is administered orally it fills the child's mouth with both an unpleasant taste and a lingering terrible aftertaste. As a result of this, children become taste averse to medications and no matter whether a given medication which has a good or bad taste, children will often refuse to take any medication at all. Many children can develop taste aversions to the point where they vomit or spit up the medication, thus rendering it useless. The process of trying to administer medications to a child becomes a struggle to get the child to open their month, swallow the medication or to even think about the concept of taking medication.

In light of the aforementioned, it would be desirable to provide an oral medication delivery mechanism that children will enjoy taking. It is well known that children enjoy candy. Therefore, it would be desirable to incorporate medications into a candy-based delivery mechanism. However, the processes required to make candy can create an in situ environment wherein the active ingredients in the medication can degrade during the production process. It would therefore be desirable to create a process to produce a gelatin and/or pectin-based chewable candy, having a given medication incorporated therein, wherein the beneficial properties of the medication is not lost due to the candy production aspect of the process. Such a medication delivery mechanism having the incorporation of drugs into gelatin and/or pectin-based candy to create a gummie or soft-chew medication delivery mechanism would be ideal to assist in children taking medication for varies aliments.

### SUMMARY OF THE GENERAL INVENTIVE CONCEPT

The following presents a simplified summary of the general inventive concept herein to provide a basic understanding of some aspects of the disclosure. This summary is not an extensive overview of the disclosure. It is not intended to restrict key or critical elements of the disclosure or to delineate the scope of the disclosure beyond that explicitly or implicitly described by the following description and claims.

In at least one exemplary embodiment of the present disclosure, the needs and objectives that will become apparent from the following description is achieved in the present disclosure which comprises a process for producing a therapeutic comestible gummie product. The process comprises providing a sugar and/or sugar alcohol and water and heating to at least 80° C. so as to produce a first mixture. A gelling agent and water are also provided and heated to at least 80° C. to produce a second mixture. The first and second mixtures are then combined to produce a slurry. The slurry is heated to at least from about 100° C. to about 130° C. to produce a cooked slurry. The cooked slurry is cooled so as to produce a gummie base having an optimal temperature for the addition of at least one active drug and the at least one active drug is substantially uniformly mixed with the gummie base. The gummie base and at least one active drug mixture is then poured into molding trays and allowed to cure.

In some exemplary embodiments, the pH of the gummie base is determined and adjusted accordingly to an optimal pH for the addition of the respective active drug or drugs.

In some exemplary embodiments, a comestible gummie product comprising a sugar and/or sugar alcohol, a gelling agent and an active drug is provided.

In some exemplary embodiments, the active drug is selected from the drug families comprising analgesics, NSAIDs, antipyretics, anti-inflammatories, antihistamines, expectorants, antitussives, decongestants and/or antibiotics.

In some exemplary embodiments, the at least one active drug is ibuprofen, acetaminophen, loratadine, cetirizine, pseudoephedrine or diphenhydramine.

In some exemplary embodiments, the process includes adding a second active drug and mixing with the gummie base. In such exemplary embodiments second active drug is ibuprofen, acetaminophen, loratadine, cetirizine, pseudoephedrine or diphenhydramine.

In another exemplary embodiment, there is provided, a process for producing a therapeutic comestible gummie product. The process comprises providing a sugar and/or sugar alcohol and water and heating to at least 80° C. so as to produce a first mixture. A gelling agent and water are also provided and heated to at least 80° C. to produce a second mixture. The first and second mixtures are then combined to produce a slurry. The slurry is heated to at least 112° C. so as to produce a cooked slurry. The cooked slurry is cooled so as to produce a gummie base having an optimal temperature for the addition an active drug, and the pH of the gummie base is determined prior to the addition of the active drug to the gummie base. The active drug is substantially uniformly mixed with the gummie base. The gummie base and the active drug mixture is then poured into molding trays and allowed to cure. By way of a process, a comestible gummie product comprising a sugar and/or sugar alcohol, a gelling agent and an active drug is also provided.

In another exemplary embodiment, there is provided, a process for producing a therapeutic comestible gummie product. The process comprises providing a sugar and/or sugar alcohol and water and heating to at least 80° C. so as to produce a first mixture. A gelling agent and water are also provided and heated to at least 80° C. to produce a second mixture. The first and second mixtures are then combined to produce a slurry. The process then involves heating the slurry to at least 112° C. to produce a cooked slurry, cooling the cooked slurry so as to produce a gummie base having a temperature of from about 70° C. to about 90° C., determining the pH of the gummie base, adjusting the pH to from about 2.5 to about 4.5, adding at least one active drug to the gummie base, substantially uniformly mixing the gummie base and the at least one active drug, pouring the gummie base and the at least one active drug mixture into molding trays, and allowing the gummie base and active drug mixture to cure. By way of a process, a comestible gummie product comprising a sugar and/or sugar alcohol, a gelling agent and an active drug is also provided.

In another exemplary embodiment, a process for producing a therapeutic comestible gummie product is provided. The process comprises providing a sugar and/or sugar alcohol and water and heating to at least 80° C. so as to produce a first mixture. A gelling agent and water are also provided and heated to at least 80° C. to produce a second mixture. The first and second mixtures are then combined to produce a slurry. The process then involves heating the slurry to at least 112° C. to produce a cooked slurry, cooling the cooked slurry so as to produce a gummie base having a temperature of from about 70° C. to about 90° C., determining the pH of the gummie base, adjusting the pH to about 3.9, adding ibuprofen to the gummie base, substantially uniformly mixing the gummie base and the ibuprofen, pouring the gummie base and the ibuprofen mixture into molding trays, and allowing the gummie base and ibuprofen mixture to cure. By way of a process, a comestible gummie product comprising a sugar and/or sugar alcohol, a gelling agent and ibuprofen is also provided.

In another exemplary embodiment a process for producing a therapeutic comestible gummie product is provided. The process comprises providing a sugar and/or sugar alcohol and water and heating to at least 80° C. so as to produce a first mixture. A gelling agent and water are also provided and heated to at least 80° C. to produce a second mixture. The first and second mixtures are then combined to produce a slurry. The slurry is heated to at least from about 100° C. to about 130° C. so as to produce a cooked slurry. The cooked slurry is cooled so as to produce a gummie base having a temperature of from about 70° C. to about 90° C. for the addition of an active drug and the active drug is substantially uniformly mixed with the gummie base. The gummie base and active drug mixture is then poured into molding trays and allowed to cure.

In another exemplary embodiment a process for producing a therapeutic comestible gummie product is provided. The process comprises providing a sugar and/or sugar alcohol and water and heating to at least 80° C. so as to produce a first mixture. A gelling agent and water are also provided and heated to at least 80° C. so as to produce a second mixture. The first and the second mixtures are combined so as to produce a slurry therefrom and the slurry is heated to at least from about 100° C. to about 130° C. to so as to produce a cooked slurry. At least one active drug is mixed in a separate vessel with a flowable substantially hydrophobic substance so as to produce an active drug and hydrophobic substance mixture. The slurry is cooled to an optimal temperature for the addition of the at least one active drug and hydrophobic substance mixture and the slurry and the hydrophobic substance with active drug mixture are substantially uniformly mixed so as to produce a gummie base having the at least one active drug and hydrophobic substance mixture incorporated therein. The gummie base with the active drug incorporated therein is then poured into molding trays and allowed to cure.

In another exemplary embodiment a process for producing a therapeutic comestible gummie product is provided. The process comprises providing a sugar and/or sugar alcohol and water and heating to at least 80° C. so as to produce a first mixture. A gelling agent and water also provided and heated to at least 80° C. so as to produce a second mixture. The first and the second mixtures are combined so as to produce a slurry therefrom and the slurry is heated to at least 112° C. to produce a cooked slurry. In a separate vessel, an active drug is mixed with a flowable substantially hydrophobic substance so as to produce an active drug and hydrophobic substance mixture. The cooked slurry is then cooled to an optimal temperature for the addition of the hydrophobic substance with active drug mixture and the active drug and hydrophobic substance mixture is mixed with cooling slurry so as to produce a gummie base having the active drug and hydrophobic substance mixture incorporated therein. The pH of the gummie base having the active drug and hydrophobic substance mixture incorporated therein is determined and the gummie base and the active drug mixture into is poured into molding trays. The gummie base and active drug mixture is then allowed to cure.

In yet another exemplary embodiment a process for producing a therapeutic comestible gummie product is provided. The process comprises providing a sugar and/or sugar alcohol and water and heating to at least 80° C. so as to produce a first mixture. A gelling agent and water are also provided and heated to at least 80° C. so as to produce a second mixture. The first and the second mixtures are combined so as to produce a slurry therefrom and the slurry is heated to at least 112° C. produce a cooked slurry. An active drug is mixed with a flowable substantially hydrophobic substance so as to produce an active drug and hydrophobic substance mixture, in a separate vessel. The cooked slurry is then cooled to a temperature of from about 70° C. to about 90° C. and the hydrophobic substance with active drug mixture are substantially uniformly mixed with the cooling slurry so as to produce a gummie base having the active drug and hydrophobic substance mixture incorporated therein. The pH of the gummie base having the active drug and hydrophobic substance mixture incorporated therein is determined and adjusted to between about 2.5 and about 4.5. The gummie base and the active drug mixture with the pH adjusted is then poured into molding trays and allowed to cure.

In some exemplary embodiments at least one flavoring agent is added to the gummie base.

In some exemplary embodiments, the process includes mixing a second active drug with the substantially hydrophobic substance.

Furthermore in some exemplary embodiments, the substantially hydrophobic substance is an ingestible oil such as coconut oil, palm oil, peanut oil or another plant-derived oil.

In some exemplary embodiments the slurry is cooled to a temperature of about 88° C. for the addition of the active drug.

### BRIEF DESCRIPTION OF THE DRAWINGS

FIG. 1 is a schematic representation of an exemplary embodiment of the process for producing the gummies having active medicinal ingredients incorporated therein of the present disclosure;

FIG. 2 is a schematic representation of an exemplary embodiment of the process for producing the gummies having active medicinal ingredients incorporated therein of the present disclosure.

### DESCRIPTION OF THE PREFERRED EMBODIMENTS

It should be understood that the disclosure is not limited in its application to the details of construction and the arrangement of components set forth in the following description or illustrated in the figures. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

Non-steroidal anti-inflammatory drugs, commonly referred to as NSAIDs, are medications or drugs which have analgesic, antipyretic or anti-inflammatory properties. As such these medications are used to reduce fever, pain and inflammation. Commonly known members of the NSAID family are aspirin, ibuprofen and naproxen and are readily available in pharmacies as over-the-counter medications; that is, accessible to the public without a prescription from a physician. Acetaminophen, another commonly used over-the-counter medication, although having analgesic and antipyretic properties is not considered an NSAID owing to the fact that it is not known to have significant anti-inflammatory properties.

In general, NSAIDs act through a mechanism of inhibiting cyclooxygenase enzymes, specifically cyclooxygense-1 (COX-1) and cycloxygenase-2 (COX-2). The inhibition of COX-1 and COX-2 inhibits the formation of prostaglandins and thromboxane from arachidonic acid. Prostaglandins serve in the body to act as a messenger in signaling for the inflammatory process to begin around a site of injury. Therefore, cyclooxygenase inhibitors act to interfere with the signaling process that leads to inflammation.

Most NSAIDs are absorbed through the stomach and intestinal mucosa. They are weak acids with a pKa of generally between 3 to 5. Furthermore, most NSAIDs are metabolized in the liver via oxidation and conjugation to inactive metabolites which are then excreted in the urine. Although the half-lives vary, ibuprofen specifically has a half-life in the body of about 2 to 3 hours.

Furthermore, most NSAIDs exist as chiral molecules and are prepared in racemic mixtures with one of the enantiomers being pharmacologically inactive. However, interestingly, typically of NSAIDs belonging to the “profen” family, isomerase enzymes exist in vivo which convert the inactive enantiomer to the active form. The activity of this isomerase varies widely between individual.

Ibuprofen has been widely used for relief of symptoms of arthritis, fever and as an analgesic. It is particularly desirable for use when its anti-inflammatory properties are required. As such, ibuprofen is a core medication in the World Health Organization's “Essential Drug List” which lists the minimum medical needs for a basic healthcare system.

Lower doses of ibuprofen are available over-the-counter is most countries, typically at doses ranging between 200 mg and 400 mg. Although the recommended dosage varies with indication, generally the oral dose is recommended from about between 5 mg to about 10 mg per kilogram of body mass in children every 1 to 2 hours to a maximum daily dose of about 800 to 1200 mg per diem.

Ibuprofen is also stable in solution; unlike other commonly used analgesics such as aspirin. However, ibuprofen is only slightly soluble in water (<1 m/ml). As such, the ibuprofen lysine salt, ibuprofen lysinate, can be used in solution to increase the water solubility. Ibuprofen lysinate allows ibuprofen to be administered intravenously.

Acetaminophen is commonly prescribed and taken by individuals as an over-the-counter medication for relief of symptoms such as pain, reducing fever, allergies, cold, cough and flu. The common adult dose of acetaminophen is from about 500 mg to about 1000 mg for an adult to maximum of about 4 grams per diem as an over-the-counter medication. Typically the children's dose of acetaminophen is about 15 mg per kilogram of body weight to a maximum dose of about 2.6 gram per diem.

Loratadine is a medication found in many over-the-counter allergy medicines. It is mainly used as an antihistamine drug, however it has also been found to be helpful in other indications such as common allergy symptom relief and psychological and neurophysical applications. Typical dosing of loratadine for adults is 10 mg administered once per day. In children the typical dosage is 5 mg administered once per day. Loratadine can also be administered as a salt, for example loratadine HBr and loratadine HCl.

Cetirizine is a selective H1 receptor inverse agonist useful in the treatment of allergies. For example cetirizine is used to treat symptoms of hay fever, angioedema and urticaria. The adult dosage and for children over the age of 6 is 10 mg administered once daily, whereas the dosage for children under the age of six is 2.5 mg administered once daily.

Pseudoephedrine is commonly used as a decongestant. It is believed its principal mechanism of action relies on its indirect action on the adrenergic receptor system wherein it has weak agonist activity at α- and β-adrenergic receptors. Pseudoephedrine causes the release of endogenous norepinephrine from storage vesicles in presynaptic neurons as its principle mode of action. The release of endogenous norepinephrine leads to vasoconstriction in the nasal mucosa and thus shrinks swollen nasal mucous membranes, thereby reducing nasal congestion. Other beneficial effects noted with pseudoephedrine may include increasing the drainage of sinus secretions, and opening of obstructed Eustachian tubes. The adult dosage, and that for children 12 years of age and older is about 60 mg every four to six hours, whereas the dosage for children 6 to 12 years of age is about 30 mg every four to six hours. Children 4 to 6 years of age have a recommended reduced dosage of about 15 mg every four to six hours. In some cases, the recommended dosage for children is about 7.5 mg every four to six hours.

Diphenhydramine is a medication found in many over-the-counter allergy medicines. It is mainly used as an antihistamine drug, however it has also been found to be helpful in other indications such as common allergy symptom relief and psychological and neurophysical applications. Typical dosing of diphenhydramine for adults is 50 mg administered once every 4 to 6 hours per day. In children the typical dosage is between 6.25 mg and 12.5 mg administered once every four to six hours per day. Diphenhydramine is typically administered as a salt, for example Diphenhydramine HCl.

The terms drug, active drug or medication, in accordance with the World Health Organization's definition of a drug, as used herein are intended to be used interchangeably and are defined as any chemical substance that, when absorbed into the body of a living organism, alters normal bodily functions. That is, for further clarity, a drug as used herein may refer to any substance with the potential to prevent or cure disease or enhance physical or mental welfare. Further, as used herein the term “drug”, in a pharmacological sense, may be used to denote any chemical agent that alters the biochemical and/or physiological processes of tissues or organisms. Hence, a drug is a substance that is, or could be, listed in a pharmacopoeia or that which is commonly referred to as a drug.

Many drugs are sensitive to the physical environment. For example, excess heat, light or moisture or sudden temperature changes can affect the stability of a drug, and many drugs lose their potency when exposed to these elements. Interaction between drugs and the environment, for example the immediate environment during the formulation manufacturing process, may affect the drug's pharmacokinetics and pharmacodynamic processes thereafter. Thus, if the proper steps to maintain the pH and temperature of a solution, mixture or slurry to which the active drug is added during the formulation process are not correct, the ultimate action of the drug upon administration could be affected. The stability and clinical effect of manufactured formulation dosage forms can be greatly compromised by seemingly negligible alterations.

Stability is defined as the extent to which a product, in this case an active drug or medication, retains, within the specified limits, the same properties and characteristics that it possessed at the time of its synthesis, throughout its period of manufacture storage and use. The primary factors that can reduce or affect the stability of drugs or medications include exposure to adverse temperatures, light, humidity, oxygen and carbon dioxide. With respect to a given dosage form, in the formulation manufacturing process, the major factors that influence drug stability, include pH, solvent system composition (percentage of “free water” and overall polarity), compatibility of anions and cations in the slurry, the ionic strength of the slurry solution, chemicals added to the slurry solution, the molecular binding of components of the slurry solution and excipients.

The degradation of many drugs in solution accelerates exponentially as the pH is increased or decreased over a specific range of pH values. Improper pH in the formulation production process is a factor most likely to cause a clinically significant loss of the drug and/or drug activity, and thus potency as a result of the hydrolysis and oxidation reactions taking place during the formulation's manufacturing process. For example, a drug in solution or suspension may be stable for a very long period of time, perhaps years, however if mixed with another liquid that changes the pH, it can degrade in minutes. It is possible that a variation of only 1 pH unit up or down from the ideal pH of a drug could decrease the drug stability by a factor of 10 or more. Therefore, it may be desirable to utilize a pH buffering system, usually a weak acid or base and their respective salts as an excipient in liquid or slurry preparations to maintain the pH in a range which would minimize drug degradation. The pH of drug solutions, in this regard, may be buffered or adjusted to achieve drug solubility while being careful to maintain the correct pH range so as not to induce drug degradation. Buffers known in the art, although not limited to, are HBr, HCl, NaOH, KOH, Citric acid, Sodium Citrate, Calcium Citrate, and Malic acid. Moreover, a multitude of other buffers and buffering salts would be acceptable to use in the formulation manufacturing process. Furthermore, the buffer and/or buffering salt choice would be dependent on the desired pH maintenance of the solution or slurry containing the active drug or medication.

For nearly all drug hydrolysis and certain drug oxidation reactions, the rate of chemical reaction increases exponentially with an increase in temperature. Therefore, as the temperature to which a drug is exposed increases, the likelihood and rate of degradation increases exponentially. Therefore, during the formulation manufacturing process, in order to maintain the active drug or medication's potency in applications where heat is required to make the desired dosage form, it is necessary to ensure that the active drug or medication is not degraded as a result of temperature.

Given the concerns of degradation of drugs in the manufacturing formulation process, care must be exercised to ensure that optimal pH and temperature are maintained in order to maintain the potency of a given drug. The following process describes such a process for producing a gelatin and/or pectin-based soft-chew or gummie drug or medication delivery mechanism. With respect to pH and temperature and active drug degradation concerns, a process for the production of a gelatin and/or pectin-based drug or medication delivery mechanism, hereinafter referred to as a “gummie” or a “soft-chew” product having an active drug ingredient incorporated therein is described. Although certain specific drugs and drug families have been aforementioned, it is contemplated that several other medications, not limited to those mentioned above, or over-the-counter medications in general may be incorporated into a gummie delivery mechanism as described herein. The process of such is hereinafter described with reference to specific details. The specific details are for the purposes of illustration only and may be variable based on the specific drug characteristics of a given medication. Therefore the actual formulation production process of a gummie having a specific active drug incorporated therein may be variable to meet the requirements of the specific drug to ensure proper activity of the drug upon administration to user within the acceptable limits of the specific drug potency.

There are several problems in producing a gummie or soft-chew product. Firstly, the product must have a pleasing look, smell, taste and texture. A notable problem associated with gummie production has been an unpleasant texture in the final product. Specifically, the gummie becomes hard or less palatable, develops uneven breakdown characteristics with mastication, and/or loses its ability to maintain its shape, and/or discolouration.

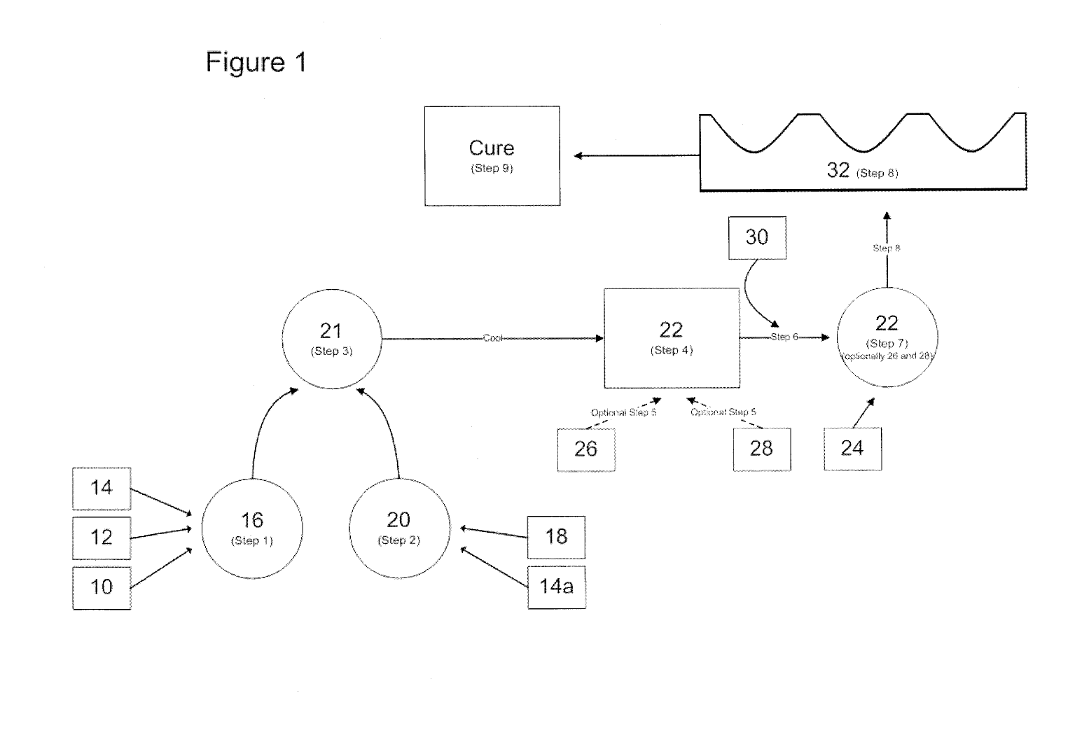

With reference now to FIG. 1. as a generalized production scheme, glucose and/or glucose granules 10, glucose syrup 12 and water 14 are combined in step 1, at 16. Other suitable sugars may be, by way of non-limiting examples, fructose, dextrose, lactose, corn syrup, maltitol, xylitol, sorbitol, mannitol or erythritol. The glucose granules 10, glucose syrup 12 and water 14 are mixed to form a glucose/water solution 16 or a first mixture 16 and heated to a temperature of about 80° C. In a separate vessel, gelatin, or alternatively pectin or a gelatin/pectin mixture or other suitable gelling agent(s) such as, for example, carragennan and konnyaku, hereinafter referred to as a gelling agent 18 is mixed with water 14a and heated to a temperature of about 80° C. to form the gelling solution 20 or second mixture 20 in step 2. Once the desired temperature of the glucose/water solution 16 and the gelling solution 20 have been reached, the two solutions are combined to form a slurry in step 3, at 21, in yet another vessel and cooked together at a temperature of from about 100° C. to about 130° C. In step 4, the now cooked slurry 21 is cooled to a temperature of from about 70° C. to about 90° C., thus forming the gummie base 22. In some preferred embodiments, the slurry 21 is cooled to about 88° C. so as to form the gummie base 22. At this point, a flavor agent or agents 26 may be optionally added. Furthermore, coloring agents 28 may also be optionally added. The optional step of adding flavoring and coloring agents is herein referred to as step 5 as is shown by dotted lines in FIG. 1. In some exemplary embodiments, while still in a flowable state, the pH of the gummie base 22 is determined and adjusted appropriately to conform to the required pH range dependent on the active drug 24 to be incorporated therein. The pH range of the flowable gummie base 22 may be adjusted to that which is pharmaceutically desired to maintain the potency of the active drug 24 using any acceptable acid or base 30 or acceptable salt thereof in step 6, such as for example, HBr, HCl, NaOH, KOH, citric acid, Sodium Citrate, Calcium Citrate and Malic acid, if required. Once the pH ranges of gummie base 22 has been adjusted in step 6 the active drug 24 is then added to the gummie base 22 in step 7. The optimal temperature of the gummie base 22 for adding and mixing in the active drug 24 in step 7 will be determined for each active drug 24 component based on the specific characteristics of the active drug 24. Furthermore, in cases where more than one active drug 24 is to be added to the gummie base 22, the gummie base 22 must be maintained at a temperature suitable to maintain the individual active drugs 24 in an active state. For example, if two active drugs are to be added to the gummie base 22, each having a different disintegration or loss of activity points based on temperature, the gummie base 22 would be maintained at a temperature suitable to incorporate the first active drug, for example the higher temperature and the first active drug mixed into the gummie base 22. The temperature of the gummie base having the first active drug incorporated therein is then lowered to a point suitable to add and mix in the second active drug. This pattern can be repeated to incorporate any number of active drugs into the gummie base 22. Once the active drug or drugs 24 have been added and substantially uniformly mixed to form a substantially homogeneous solution or mixture with the gummie base 22, the still flowable gummie base 22 is poured into molding trays 32 in step 8. In step 9, the molding trays 32 with the gummie base 22 having the active drug or drugs 24 incorporated therein are allowed to cure for a given period of time. Curing can be defined as the solidification process to a pliable soft-chew product which is capable of breakdown with mastication and/or following ingestion by a user. The curing in step 9 has several variables, such as curing time, relative humidity and curing temperature which may be different depending on the resultant composition of the gummie base 22 and active drugs incorporated therein. The curing time on average will be 48 hrs, however this may vary dependant on the composition of the gummie base 22 with the active drug 24 incorporated therein. Furthermore, on average the curing temperature begins at about 32° C. to 40° C., and then is lowered to a temperature of 30° C. with from about 0% to about 50% relative humidity. Preferably, the relative humidity for curing is from about 0% to about 40%. More preferably, the relative humidity for curing is from about 0% to about 30%. In preferred embodiments the ambient temperature for curing is maintained at from about 36° C. to about 38° C. with a relative humidity of from about 20% to about 25%. Most preferably, the relative humidity for curing is about 21% and the ambient temperature is maintained at about 37.2° C.

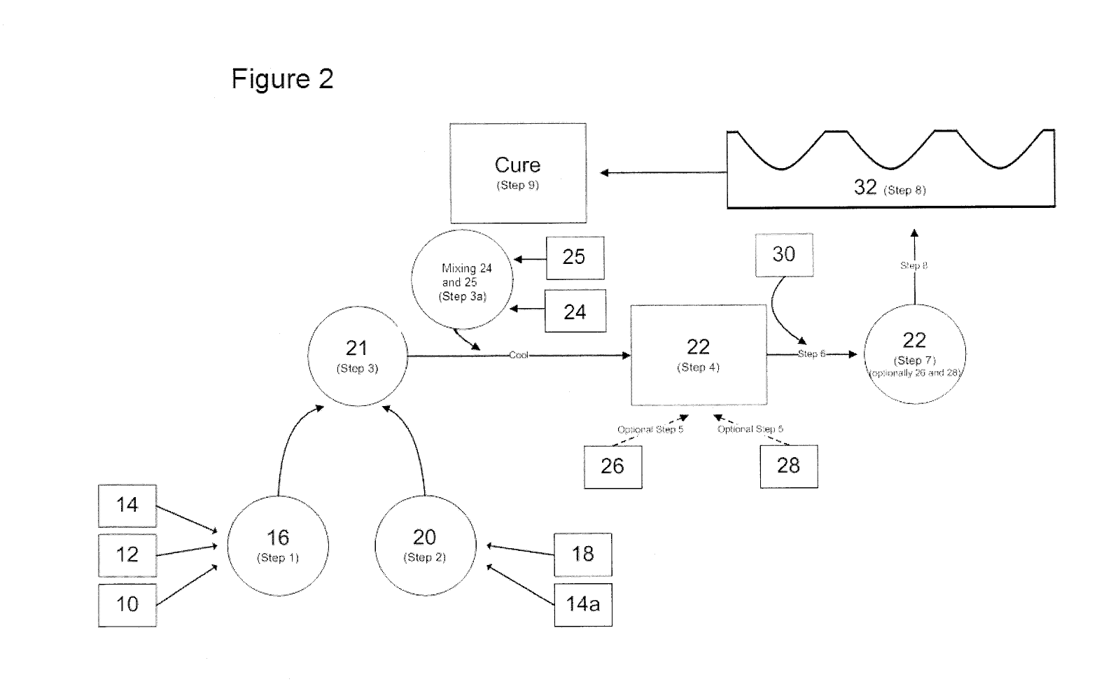

With reference to FIG. 2, in another embodiment, it may by necessary to first mix the active drug 24 with an ingestible and flowable substantially hydrophobic substance, denoted by 25 and mix the active drug 24 and hydrophobic substance 25 mixture into cooked slurry 21 at an acceptable point during the cooling process as shown at step 3a. The hydrophobic substance 25, may be, for example, coconut oil, palm oil, peanut oil or other suitable plant-derived oil. The active drug 24 is mixed with the hydrophobic substance or oil 25 so as to improve blend consistency and improve solubility of the active drug 24 prior to mixing with the cooling slurry 21. Furthermore, it is understood that by first mixing the active drug 24 with a hydrophobic substance 25, the active drug 24 may be substantially encapsulated or may be protected in some manner and thus, in a sense, also be protected from substantial degradation during the reminder of the process while improving the blend consistency and improving the solubility of the active drug 24.

For example, an exemplary embodiment, as show in FIG, 2, may proceed substantially as noted above with reference to FIG. 1. That is, FIG. 2 shows a generalized production scheme, wherein glucose and/or glucose granules 10, glucose syrup 12 and water 14 are combined in step 1, at 16. Other suitable sugars may be as noted above with reference to FIG. 1. The glucose granules 10, glucose syrup 12 and water 14 are mixed to form a glucose/water solution 16 or a first mixture 16 and heated to a temperature of about 80° C. In a separate vessel, a gelling agent 18 is mixed with water 14a and heated to a temperature of about 80° C. to form the gelling solution 20 or second mixture 20 in step 2. Once the desired temperature of the glucose/water solution 16 and the gelling solution 20 have been reached, the two solutions are combined to form a slurry in step 3, at 21, in yet another vessel and cooked together at a temperature of from about 100° C. to about 130° C. A flowable substantially hydrophobic substance 25 is then mixed in a separate vessel with at least one active drug 24. Although not shown in FIG. 2, in some exemplary embodiments, a second active drug may also be mixed with the substantially hydrophobic substance 25. In step 3a, the active drug 24 and hydrophobic substance 25 mixture is then added to the slurry 21 and substantially homogenously mixed into the slurry 21 during the cooling process at a point when the temperature of the slurry 21 is suitable so not to cause substantial degradation of the active drug 24. Such a temperature is determined for each active drug 24 based on the specific characteristics of the drug. In step 4, the now cooked slurry 21 having the active drug 24 incorporated therein is further cooled to a temperature of from about 70° C. to about 90° C., thus forming the gummie base 22 with the at least one active drug incorporated therein. In some preferred embodiments, the slurry 21 is cooled to about 88° C. so as to form the gummie base 22 with the active drug 24 incorporated therein. Similar to the generalized production scheme of FIG. 1, at this point, a flavor agent or agents 26 may be optionally added. Furthermore, coloring agents 28 may also be optionally added. The optional step of adding flavoring 26 and coloring 28 agents is herein referred to as step 5 as is shown by dotted lines in FIG. 2. While still in a flowable state, the pH of the gummie base 22 is determined and adjusted appropriately to conform to the required pH range dependent on the active drug 24 to be incorporated therein. The pH range of the flowable gummie base 22 may be adjusted to that which is pharmaceutically desired to maintain the potency of the active drug 24 using any acceptable acid or base 30 or acceptable salt thereof in step 6, such as for example, HBr, HCl, NaOH, KOH, citric acid, Sodium Citrate, Calcium Citrate and Malic acid, if required. Once the pH ranges of gummie base 22 has been adjusted as may be required in step 6 the gummie base 22 with the active drug 24 incorporated therein is poured into molding trays 32 in step 8. In step 9, the molding trays 32 with the gummie base 22 having the active drug or drugs 24 incorporated therein are allowed to cure for a given period of time. The curing has been discussed above and is provided as part of the process so as to form a pliable soft-chew product which is capable of breakdown with mastication and/or following ingestion by a user. Furthermore, variables involved in the curing step 9 are also discussed above with reference to FIG. 1. However, the curing time on average will be 48 hrs, although this may vary dependant on the composition of the gummie base 22 with the active drug 24 incorporated therein. Furthermore, on average the curing temperature begins at about 32° C. to 40° C., and then is lowered to a temperature of 30° C. with from 0% to about 50% relative humidity. Preferably, the relative humidity for curing is from about 0% to 40%. More preferably, the relative humidity for curing is from 0% to about 30%. In preferred embodiments the ambient temperature for curing is maintained at from about 36° C. to about 38° C. with a relative humidity of from about 20% to about 25%. Most preferably, the relative humidity for curing is about 21% and the ambient temperature is maintained at about 37.2° C.

The optimal temperature of the gummie base for the addition of the active drug as used herein is defined as the temperature range for which the active drug does not decay or a temperature range at which there is no significant loss of functionality. For example, in the case of ibuprofen, the optimal temperature range is between 70° C. and 110° C. Preferably, the optimal temperature range for ibuprofen is from about 70° C. to about 80° C. Ideally, the optimal temperature for ibuprofen is about 76° C. Loratadine melts at temperatures above 111° C. and decomposes at its boiling point. Therefore the optimal temperature for Loratadine is less than 111° C. and preferably the optimal temperature range is from about 70° C. to about 100° C. Ideally, the optimal temperature for Loratadine is about 80° C. Pseudoephedrine melts at temperatures above 116° C. Therefore, the optimal temperature range for pseudoephedrine is between from about 70° C. to about 100° C. Ideally, the optimal temperature for pseudoephedrine is about 80° C. Cetirizine melts at temperatures above 110° C. Therefore, the optimal temperature range for cetirizine is between from about 70° C. to about 100° C. Ideally, the optimal temperature for cetirizine is about 80° C. Acetaminophen melts at temperatures above 170° C. Therefore, the optimal temperature range for acetaminophen is between from about 70° C. to about 100° C. Ideally, the optimal temperature for acetaminophen is about 80° C. Diphenhydramine melts at about 166° C. to about 169° C., the optimal temperature range is between 70° C. to about 100° C. Ideally the optimal temperature for Diphenhydramine is about 76° C.

### EXAMPLES

### Example 1

With reference to an individual serving, the following production scheme is performed in the manufacture of an ibuprofen-containing gummie product. About 0.7 g to about 1.3 g of glucose, about 0.1 to about 0.3 g of water and about 0.6 g to about 1.3 g of glucose syrup is combined in a vessel and heated to a temperature of about 80° C. thus forming a first mixture. In a separate vessel, about 0.1 g to about 0.2 g of gelatin, about 0.08 g to about 0.1 g of pectin are mixed with a suitable amount of water and heated to a temperature of about 80° C. thus forming a second mixture. The aforementioned first and second mixtures once heated to about 80° C. are combined to form slurry and heated to a temperature of about 112° C. thus producing a cooked slurry. The cooked slurry is then cooled so as to produce a gummie base having a temperature of from about 70° C. to about 90° C. thus forming a gummie base. Once the gummie base is cooled to a temperature of from about 70° C. to about 90° C., from about 0.025 g to about 0.04 g of citric acid, from about 0.002 g to about 0.004 g, about 0.003 g to about 0.005 g of malic acid, from about 0.002 g to about 0.005 g of colouring agents, and from about 0.0001 g to about 0.006 g of flavoring agents are added and substantially uniformly mixed into the gummie base. In the present exemplary embodiment, about 0.004 g per individual serving of flavoring agent is added and mixed into the gummie base. The pH of the gummie base at this point is determined and is adjusted using conventional means to be between from about 2.5 to about 4.5. Preferably the pH is adjusted to be between from about 3 to about 4.2. Ideally, the pH of the gummie base is adjusted to about 3.9. Once the pH has been adjusted, and the temperature is ideally maintained at about 76° C., from about 1 mg to about 120 mg of ibuprofen is added and substantially uniformly mixed into the gummie base. Ideally, from about 25 mg to about 75 mg of ibuprofen is added to the gummie base. Optimally, about 50 mg ibuprofen is added to the gummie base. This gummie base having ibuprofen substantially uniformly mixed therein is poured into molding trays and allowed to cure at about 21% relative humidity for about 48 hrs or until cured with an ambient temperature of about 37.2° C.

In this example, the ranges are given for a single ibuprofen-containing gummie product, however in a production setting the relative amounts of each ingredient is increased proportionally to satisfy the desired quantity of individual gummie products. The final weight of each gummie product containing ibuprofen when produced in a manufacturing setting is from about 1 g to about 5 g and preferably from about 1.5 g to about 2.5 g.

### Example 2

With reference to an individual serving, the following production scheme is performed in the manufacture of a loratadine-containing gummie product. About 0.9 g to about 1.0 g of glucose, about 0.1 g to about 0.2 g of water and about 0.6 g to about 0.7 g of glucose syrup is combined in a vessel and heated to a temperature of about 80° C. thus forming a first mixture. In a separate vessel, about 0.1 g to about 0.2 g of gelatin, about 0.08 g to about 0.1 g of pectin are mixed with a suitable amount of water and heated to a temperature of about 80° C. thus forming a second mixture. The aforementioned first and second mixtures once heated to about 80° C. are combined to form slurry and heated to a temperature of about 112° C. thus producing a cooked slurry. The cooked slurry is then cooled so as to produce a gummie base having temperature of from about 70° C. to about 90° C. thus forming a gummie base. Once the gummie base is cooled to a temperature of from about 70° C. to about 90° C., from about 0.025 to about 0.03 g of citric acid, from about 0.002 g to about 0.004 g, about 0.003 g to about 0.005 g of malic acid, from about 0.002 g to about 0.005 g of colouring agents, and from about 0.0001 g to about 0.006 g of flavoring agents are added and substantially uniformly mixed into the gummie base. The pH of the gummie base at this point is determined and is adjusted using convention means to be between from about 2.5 to about 4.5. Preferably the pH is adjusted to be between from about 3.0 to about 4.2. Ideally, the pH of the gummie base is adjusted to about 3.9. Once the pH has been adjusted the temperature is ideally maintained at about 76° C., and from about 5 mg to about 50 mg of loratadine is added and substantially uniformly mixed into the gummie base. Optimally, about 10 mg of loratadine is added to the gummie base. This gummie base having loratadine substantially uniformly mixed therein is poured into molding trays and allowed to cure at about 21% relative humidity for about 48 hrs or until cured with an ambient temperature of about 37.2° C.

In this example, the ranges are given for a single loratadine-containing gummie product; however in a production setting the relative amounts of each ingredient is increased proportionally to satisfy the desired quantity of individual gummie products. The final weight of each gummie product containing loratadine when produced in a manufacturing setting is from about 1 g to about 5 g and preferably from about 1.5 g to about 2.5 g.

### Example 3

With reference to an individual serving, the following production scheme is performed in the manufacture of a cetirizine-containing gummie product. About 0.9 g to about 1.0 g of glucose, about 0.1 g to about 0.2 g of water and about 0.6 g to about 0.7 g of glucose syrup is combined in a vessel and heated to a temperature of about 80° C. thus forming a first mixture. In a separate vessel, about 0.1 g to about 0.2 g of gelatin, about 0.08 g to about 0.1 g of pectin are mixed with a suitable amount of water and heated to a temperature of about 80° C. thus forming a second mixture. The aforementioned first and second mixtures once heated to about 80° C. are combined to form slurry and heated to a temperature of about 112° C. thus producing a cooked slurry. The cooked slurry is then cooled so as to produce a gummie base having of from about 70° C. to about 90° C. thus forming a gummie base. Once the gummie base is cooled to a temperature of from about 70° C. to about 90° C., from about 0.025 g to about 0.03 g of citric acid, from about 0.002 g to about 0.004 g, about 0.003 g to about 0.005 g of malic acid, from about 0.002 g to about 0.005 g of colouring agents, and from about 0.0001 g to about 0.006 g of flavoring agents are added and substantially uniformly mixed into the gummie base. The pH of the gummie base at this point is determined and is adjusted using convention means to be between from about 2.5 to about 4.5. Preferably the pH is adjusted to be between from about 3 to about 4.2. Ideally, the pH of the gummie base is adjusted to about 3.9. Once the pH has been adjusted and the temperature is ideally maintained at about 76° C., from about 1 mg to about 10 mg of cetirizine is added and substantially uniformly mixed into the gummie base. Optimally, about 2.5 mg cetirizine is added to the gummie base. This gummie base having cetirizine substantially uniformly mixed therein is poured into molding trays and allowed to cure at about 21% relative humidity for about 48 hrs or until cured with an ambient temperature of about 37.2° C.

In this example, the ranges are given for a single cetirizine-containing gummie product, however in a production setting the relative amounts of each ingredient is increased proportionally to satisfy the desired quantity of individual gummie products. The final weight of each gummie product containing cetirizine when produced in a manufacturing setting is from about 1 g to about 5 g and preferably from about 1.5 g to about 2.5 g.

### Example 4

With reference to an individual serving, the following production scheme is performed in the manufacture of an acetaminophen-containing gummie product. About 0.9 g to about 1.0 g of glucose, about 0.1 g to about 0.2 g of water and about 0.6 g to about 0.7 g of glucose syrup is combined in a vessel and heated to a temperature of about 80° C. thus forming a first mixture. In a separate vessel, about 0.1 g to about 0.2 g of gelatin, about 0.08 g to about 0.1 g of pectin are mixed with a suitable amount of water and heated to a temperature of about 80° C. thus forming a second mixture. The aforementioned first and second mixtures once heated to about 80° C. are combined to form slurry and heated to a temperature of about 112° C. thus producing a cooked slurry. The cooked slurry is then cooled so as to produce a gummie base having temperature of from about 70° C. to about 90° C. thus forming a gummie base. Once the gummie base is cooled to a temperature of from about 70° C. to about 90° C. from about 0.025 g to about 0.03 g of citric acid, from about 0.002 g to about 0.004 g, about 0.003 g to about 0.005 g of malic acid, from about 0.002 g to about 0.005 g of colouring agents, and from about 0.0001 g to about 0.006 g of flavoring agents are added and substantially uniformly mixed into the gummie base. The pH of the gummie base at this point is determined and is adjusted using conventional means to be between about 2.5 and about 4.5. Preferably the pH is adjusted to be between from about 3 to about 4.2. Ideally, the pH of the gummie base is adjusted to about 3.9. Once the pH has been adjusted and the temperature is ideally maintained at about 76° C., from about 25 mg to about 120 mg of acetaminophen is added and substantially uniformly mixed into the gummie base. In some exemplary embodiments, 100 mg acetaminophen is added to the gummie base. However in some other exemplary embodiments, about 80 mg per individual serving is added to the gummie base. This gummie base having acetaminophen substantially uniformly mixed therein is poured into molding trays and allowed to cure at about 21% relative humidity for about 48 hrs or until cured with an ambient temperature of about 37.2° C.

In this example, the ranges are given for a single acetaminophen-containing gummie product, however in a production setting the relative amounts of each ingredient is increased proportionally to satisfy the desired quantity of individual gummie products. The final weight of each gummie product containing acetaminophen when produced in a manufacturing setting is from about 1 g to about 5 g and preferably from about 1.5 g to about 2.5 g.

### Example 5

With reference to an individual serving, the following production scheme is performed in the manufacture of a pseudoephedrine-containing gummie product. About 0.9 g to about 1.0 g of glucose, about 0.1 g to about 0.2 g of water and about 0.6 g to about 0.7 g of glucose syrup is combined in a vessel and heated to a temperature of about 80° C. thus forming a first mixture. In a separate vessel, about 0.1 g to about 0.2 g of gelatin, about 0.08 g to about 0.1 g of pectin are mixed with a suitable amount of water and heated to a temperature of about 80° C. thus forming a second mixture. The aforementioned first and second mixtures once heated to about 80° C. are combined to form slurry and heated to a temperature of about 112° C. thus producing a cooked slurry. The cooked slurry is then cooled so as to produce a gummie base having a temperature of from about 70° C. to about 90° C. thus forming a gummie base. Once the gummie base is cooled to a temperature of from about 70° C. to about 90° C., from about 0.025 g to about 0.03 g of citric acid, from about 0.002 g to about 0.004 g, about 0.003 g to about 0.005 g of malic acid, from about 0.002 g to about 0.005 g of colouring agents, and from about 0.0001 g to about 0.006 g of flavoring agents are added and substantially uniformly mixed into the gummie base. The pH of the gummie base at this point is determined and is adjusted using conventional means to be between about 2.5 and about 4.5. Preferably the pH is adjusted to be between from about 3 to about 4.2. Ideally, the pH of the gummie base adjusted to about 3.9. Once the pH has been adjusted and the temperature is ideally maintained at about 76° C., from about 5 mg to about 50 mg of pseudoephedrine is added and substantially uniformly mixed into the gummie base. In some exemplary embodiments, about 15 mg pseudoephedrine per individual serving is added to the gummie base. However in other exemplary embodiments, about 7.5 mg of pseudoephedrine per individual serving is added to the gummie base. This gummie base having pseudoephedrine substantially uniformly mixed therein is poured into molding trays and allowed to cure at about 21% relative humidity for about 48 hrs or until cured with an ambient temperature of about 37.2° C.

In this example, the ranges are given for a single pseudoephedrine-containing gummie product, however in a production setting the relative amounts of each ingredient is increased proportionally to satisfy the desired quantity of individual gummie products. The final weight of each gummie product containing pseudoephedrine when produced in a manufacturing setting is from about 1 g to about 5 g and preferably from about 1.5 g to about 2.5 g.

### Example 6

With reference to an individual serving, the following production scheme is performed in the manufacture of a diphenhydramine-containing gummie product. About 0.9 g to about 1.0 g of glucose, about 0.1 g to about 0.2 g of water and about 0.6 g to about 0.7 g of glucose syrup is combined in a vessel and heated to a temperature of about 80° C. thus forming a first mixture. In a separate vessel, about 0.1 g to about 0.2 g of gelatin, about 0.08 g to about 0.1 g of pectin are mixed with a suitable amount of water and heated to a temperature of about 80° C. thus forming a second mixture. The aforementioned first and second mixtures once heated to about 80° C. are combined to form slurry and heated to a temperature of about 112° C. thus producing a cooked slurry. The cooked slurry is then cooled so as to produce a gummie base having temperature of from about 70° C. to about 90° C. thus forming a gummie base. Once the gummie base is cooled to a temperature of from about 70° C. to about 90° C., from about 0.025 to about 0.03 g of citric acid, from about 0.002 g to about 0.004 g, about 0.003 g to about 0.005 g of malic acid, from about 0.002 g to about 0.005 g of colouring agents, and from about 0.0001 g to about 0.006 g of flavoring agents are added and substantially uniformly mixed into the gummie base. The pH of the gummie base at this point is determined and is adjusted using convention means to be between from about 2.5 to about 4.5. Preferably the pH is adjusted to be between from about 3.0 to about 4.2. Ideally, the pH of the gummie base is adjusted to about 3.9. Once the pH has been adjusted the temperature is ideally maintained at about 76° C., and from about 2 mg to about 50 mg of diphenhydramine is added and substantially uniformly mixed into the gummie base. Optimally, 6.25 mg of diphenhydramine is added to the gummie base. This gummie base having the diphenhydramine substantially uniformly mixed therein is poured into molding trays and allowed to cure at about 21% relative humidity for about 48 hrs or until cured with an ambient temperature of about 37.2° C.

In this example, the ranges are given for a single diphenhydramine-containing gummie product, however in a production setting the relative amounts of each ingredient is increased proportionally to satisfy the desired quantity of individual gummie products. The final weight of each gummie product containing pseudoephedrine when produced in a manufacturing setting is from about 1 g to about 5 g and preferably from about 1.5 g to about 2.5 g.

### Example 7

With reference to an individual serving, the production scheme is performed in manufacture of a diphenhydramine-containing gummie product. About 0.9 g to about 1.0 g of glucose, about 0.1 g to about 0.2 g of water and about 0.6 g to about 0.7 g of glucose syrup is combined in a vessel and heated to a temperature of about 80° C. thus forming a first mixture. In a separate vessel, about 0.1 g to about 0.2 g of gelatin, about 0.08 g to about 0.1 g of pectin are mixed with a suitable amount of water and heated to a temperature of about 80° C. thus forming a second mixture. The aforementioned first and second mixtures once heated to about 80° C. are combined to form slurry and heated to a temperature of about 112° C. thus producing a cooked slurry. In some exemplary embodiments, about 2 mg to about 50 mg of diphenhydramine and from about 0.01 ml to about 0.1 ml of coconut oil are combined in separate vessel. However, other suitable oils may be a plant-derived oil, peanut oil or palm oil. The diphenhydramine and coconut oil is combined in a ratio so as to produce a consistent blend for incorporation into an individual gummie serving. In a desired embodiment, about 6.25 mg of diphenhydramine is mixed the coconut oil per individual serving. The slurry is then allowed to cool and the coconut oil with the diphenhydramine incorporated therein is added to the cooling slurry once the temperature of the cooling slurry suitable to as not to cause substantial degradation of the active compound, in this case diphenhydramine. The slurry with diphenhydramine and coconut oil incorporated therein is substantially uniformly mixed and further cooled to a temperature of about 70° C. to about 90° C. thus forming a gummie base with the diphenhydramine incorporated therein. Once the gummie base is cooled to a temperature of from about 70° C. to about 90° C., from about 0.025 g to about 0.03 g of citric acid, from about 0.002 g to about 0.004 g, about 0.003 g to about 0.005 g of malic acid, from about 0.002 g to about 0.005 g of colouring agents, and from about 0.0001 g to about 0.006 g of flavoring agents are added and substantially uniformly mixed into the gummie base. The pH of the gummie base at this point is determined and is adjusted using conventional means to be between about 2.5 and about 4.5. Preferably the pH is adjusted to be between from about 3 to about 4.2. Ideally, the pH of the gummie base adjusted to about 3.9. This gummie base having diphenhydramine substantially uniformly mixed therein is poured into molding trays and allowed to cure at about 21% relative humidity for about 48 hrs or until cured with an ambient temperature of about 37.2° C.

In this example, the ranges are given for a single diphenhydramine-containing gummie product, however in a production setting the relative amounts of each ingredient is increased proportionally to satisfy the desired quantity of individual gummie products. The final weight of each gummie product containing pseudoephedrine when produced in a manufacturing setting is from about 1 g to about 5 g and preferably from about 1.5 g to about 2.5 g.

### Example 8

With reference to an individual serving, and exemplary another embodiment, the production scheme is performed in manufacture of an acetaminophen-containing gummie product. About 0.9 g to about 1.0 g of glucose, about 0.1 g to about 0.2 g of water and about 0.6 g to about 0.7 g of glucose syrup is combined in a vessel and heated to a temperature of about 80° C. thus forming a first mixture. In a separate vessel, about 0.1 g to about 0.2 g of gelatin, about 0.08 g to about 0.1 g of pectin are mixed with a suitable amount of water and heated to a temperature of about 80° C. thus forming a second mixture. The aforementioned first and second mixtures once heated to about 80° C. are combined to form slurry and heated to a temperature of about 112° C. thus producing a cooked slurry. From about 0.01 ml to about 0.1 ml of coconut oil and about 100 mg of acetaminophen are combined in separate vessel. However, other suitable oils may be a plant-derived oil, peanut oil or palm oil for mixing the acetaminophen in ratios for consistent blending of the active drug and oil. In some other exemplary embodiments, about 80 mg per individual serving of acetaminophen is combined with the oil so as to produce an individual serving. The slurry is then allowed to cool and the coconut oil with the acetaminophen incorporated therein is added to the cooling slurry once the temperature of the cooling slurry suitable to as not to cause substantial degradation of the acetaminophen. The slurry with acetaminophen and coconut oil incorporated therein is substantially uniformly mixed and further cooled to a temperature of about 70° C. to about 90° C. thus forming a gummie base with acetaminophen incorporated therein. Once the gummie base is cooled to a temperature of from about 70° C. to about 90° C., from about 0.025 g to about 0.03 g of citric acid, from about 0.002 g to about 0.004 g, about 0.003 g to about 0.005 g of malic acid, from about 0.002 g to about 0.005 g of colouring agents, and from about 0.0001 g to about 0.006 g of flavoring agents are added and substantially uniformly mixed into the gummie base. The pH of the gummie base at this point is determined and is adjusted using conventional means to be between about 2.5 and about 4.5. Preferably the pH is adjusted to be between from about 3 to about 4.2. Ideally, the pH of the gummie base adjusted to about 3.9. This gummie base having acetaminophen substantially uniformly mixed therein is poured into molding trays and allowed to cure at about 21% relative humidity for about 48 hrs or until cured with an ambient temperature of about 37.2° C.

In this example, the ranges are given for a single acetaminophen-containing gummie product, however in a production setting the relative amounts of each ingredient is increased proportionally to satisfy the desired quantity of individual gummie products. The final weight of each gummie product containing pseudoephedrine when produced in a manufacturing setting is from about 1 g to about 5 g and preferably from about 1.5 g to about 2.5 g.

1-82. (canceled)

83. A process for producing a therapeutic comestible gummie product, the process comprising:
providing a sugar and/or sugar alcohol and water and heating to at least 80° C. so as to produce a first mixture;
providing a gelling agent and water and heating to at least 80° C. so as produce a second mixture;
combining the first and the second mixture to as to produce a slurry therefrom;
heating the slurry to at least from about 100° C. to about 130° C. to produce a cooked slurry;
cooling the cooked slurry so as to produce a gummie base having an optimal temperature for the addition of at least one active drug;
adding the at least one active drug to the gummie base;
substantially uniformly mixing the gummie base and the at least one active drug;
pouring the gummie base and the at least one active drug mixture into molding trays; and
allowing the gummie base and the at least one active drug mixture to cure.

84. The process as defined in claim 83, wherein the sugar is glucose, fructose, dextrose, lactose, corn syrup, maltitol, xylitol, sorbitol, mannitol or erythritol.

85. The process as defined in claim 83, wherein the gelling agent is gelatin, pectin, carragennan or konnyaku.

86. The process as defined in claim 83, wherein the slurry is heated to about 112° C.

87. The process as defined in claim 83, wherein the at least one active drug is an analgesic, an NSAID, an antipyretic, an anti-inflammatory, an antihistamine, an expectorant, an antitussive, a decongestant or an antibiotic.

88. The process as defined in claim 87, wherein the at least one active drug is ibuprofen, acetaminophen, loratadine, cetirizine, pseudoephedrine or diphenhydramine.

89. The process as defined in claim 83, including mixing a second active drug with the gummie base.

90. The process as defined in claim 89, wherein the second active drug is ibuprofen, acetaminophen, loratadine, cetirizine, pseudoephedrine or diphenhydramine.

91. The process as defined in claim 83, including determining the pH of the gummie base prior to mixing in the at least one active drug.

92. The process as defined in claim 91, including adjusting the pH of the gummie base to a pH suitable for the addition of the at least one active drug.

93. The process as defined in claim 92, wherein the pH of the gummie base is adjust so as to be between from about 2.5 to about 4.5.

94. The process as defined in claim 93, wherein the pH of the gummie base is adjusted to about 3.9 and the at least one active drug is ibuprofen.

95. The process as defined in claim I wherein the slurry is cooled to a temperature of from about 70° C. to about 90° C.

96. The process as defined in claim 95, wherein the slurry is cooled to a temperature of about 88° C.

97. The process as defined in claim 83, further comprising mixing the at least one active drug with a flowable hydrophobic substance so as to produce an active drug and hydrophobic substance mixture prior to mixing with the gummie base.

98. The process as defined in claim 97, further comprising adding a second active drug with the at least one active drug and hydrophobic substance.

99. The process as defined in claim 98, wherein the second active drug is ibuprofen, acetaminophen, loratadine, cetirizine, pseudoephedrine or diphenhydramine.

100. The process as defined in claim 99, wherein the flowable hydrophobic substance is an ingestible oil.

101. The process as defined in claim 100, wherein the ingestible oil is coconut oil, palm oil, peanut oil or another plant-derived oil.

102. The process as defined in claim 97, including adjusting the pH of the gummie base to a pH suitable for the addition of the at least one active drug.

103. The process as defined in claim 102, wherein the pH of the gummie base is adjusted so as to be between from about 2.5 to about 4.5.

104. The process as defined in claim 103, wherein the pH of the gummie base is adjusted to about 3.9 and the at least one active drug is diphenhydramine.

105. The process as defined in claim 104, wherein the amount of diphenhydramine is from about 2 mg to about 50 mg per serving and the amount of coconut oil is from about 0.01 ml to about 0.1 ml per serving.
